(19) United States
(12) Patent Application Publication
(10) Pub. No.: US 2006/0149057 A1
(43) Pub. Date: Jul. 6, 2006

(54) Method of purifying macrolides

(76) Inventors: Vilmos Keri, Debrecen (HU); Zoltan Czovek, Debrecen (HU)

Correspondence Address: KENYON & KENYON LLP, ONE BROADWAY, NEW YORK, NY, 10004, US

(21) Appl. No.: 11/317,152

(22) Filed: Dec. 22, 2005

Related U.S. Application Data
(60) Provisional application No. 60/638,628, filed on Dec. 22, 2004.
(60) Provisional application No. 60/638,815, filed on Dec. 23, 2004.

(51) Int. Cl. C07D 267/22; C07D 281/18

(52) U.S. Cl. 540/456; 540/457

(57) ABSTRACT

A method for purifying macrolide is provided in which a loading charge of macrolide is placed in juxtaposition with a bed of wet sorption resin, the loading charge and bed are eluted at a temperature greater than 30° C. with an eluent of an organic solvent selected from the group consisting of THF, acetonitrile, n-propyl alcohol, iso-propyl alcohol, ethyl alcohol, and acetone, the heart cut of the eluent is collected, and purified macrolide is collected.

### RELATED APPLICATIONS

This application claims the benefits of U.S. Provisional Applications Ser. No. 60/638,628, filed Dec. 22, 2004, and Ser. No. 60/638,815, filed Dec. 23, 2004, the contents of which are incorporated herein by reference in their entirety.

The present invention relates to a method of purifying macrolides, especially tacrolimus, ascomycin, sirolimus, everolimus, or pimecrolimus, by a separation method using sorption resins at an elution temperature of more than about 30° C.

### BACKGROUND OF THE INVENTION

Macrolides are multi-membered lactone rings having one or more deoxy sugars as substituents. Erythromycin, azithromycin, and clarithromycin are macrolides that have bacteriostatic and/or bactericidal activity.

Tacrolimus (FK 506) is also a macrolide antibiotic that is also an immunosuppressive agent. More potent than cyclosporin, tacrolimus reportedly has a selective inhibitory effect on T-lymphocytes.

Pimecrolimus is a macrolactam and a ascomycin derivative that reportedly inhibits production of pro-inflammatory cytokines by T cells and mast cells. The Merck Index 1331 (Maryadele J. O'Neil et al. eds., 13th ed. 2001). Pimecrolimus is reportedly used as an immunosuppressant. Id.

Sirolimus, another macrolide, is reported to be an immunosuppressant. Sirolimus has been administered with cyclosporin and corticosteroids after transplantation to avoid graft rejection. Martindale: The Complete Drug Reference 568 (Sean C. Sweetman ed., Pharmaceutical Press 33rd ed. 2002).

Everolimus, a derivative of sirolimus, is reported to be an immunosuppressant used in organ transplantation. Martindale at 539.

The macrolides are typically obtained by fermentation, although synthetic routes to some are known. Macrolides, as obtained, typically contain several impurities that can be detected by various means, for example high-pressure liquid chromatography (HPLC). Presence of impurities in a pharmaceutical compound is undesirable, and health authorities in many jurisdictions (e.g. the Food and Drug Administration in the United States) have established guidelines relating to acceptable levels of impurities in pharmaceuticals. The need for and commercial utility of methods of reducing the level of impurities in any pharmaceutical are self-evident.

U.S. Pat. Nos. 4,894,366, 6,576,135, 6,881,341 and 6,492,513, for example, disclose purification processes of tacrolimus. There is a need for other, more efficient methods for purification of macrolides.

### SUMMARY OF THE INVENTION

In one embodiment, the present invention provides a chromatographic method for purifying macrolides. The method comprises providing a loading charge of a macrolide, loading a loading charge of the macrolide onto a bed of sorption resin and eluting with an eluent that contains at least one organic solvent selected from the group consisting of THF, acetonitrile, n-propyl alcohol, iso-propyl alcohol, ethyl alcohol, and acetone and water, at a temperature greater than about 30° C., to about the boiling temperature of the solvent to obtain an effluent, collecting the main fraction of the effluent, and recovering the macrolide.

In another aspect, the present invention relates to macrolides prepared by the method described above, especially tacrolimus, ascomycin, sirolimus (rapamycin), everolimus, and pimecrolimus.

### DETAILED DESCRIPTION OF THE INVENTION

As used herein, the term “reduced pressure” refers to a pressure of less than about 760 mm Hg. Also, as used herein, the term “area percent” refers to area percent of HPLC chromatograms obtained by the method of the invention.

As used herein, the term anti-solvent refers to a substance, normally liquid at ambient temperature, in which macrolide is at best sparingly soluble.

As used herein, the term “impurity” relates to any compound having a different retention time than the desired macrolide. The different retention time may be measured, for example, by the HPLC method described herein below.

As used herein, the terms RRT0.95 and RRT1.25 refer to ascomycin and dihydrotacrolimus, respectively, which are impurities in tacrolimus, having relative retention times (to tacrolimus) of about 0.95 and 1.25 in HPLC analysis, such as the one described herein below.

As used herein in connection with mixtures or combinations of liquids, the term volume percent or percent-by-volume (vol-%) refers to volume fraction calculated as follows (illustrated for species A):
vol-%_{A}=Wt_{A}×ρ_{A}/(Wt_{A}×ρ_{A}+Wt_{B}×ρ_{B})
where: Wt_{A} and Wt_{B} are the weights in grams of species A and B, respectively, and ρ_{A} and ρ_{B} are the densities, in g/ml of species A and B, respectively.

By applying a chromatographic method for separating impurities from macrolides at a temperature greater than about 30° C. to about the boiling temperature of the solvent (included in the eluent), a much better purity may be achieved.

In one embodiment, the present invention provides a chromatographic method for purifying macrolides (i.e. for reducing the level of impurities in a macrolide). The method comprises providing a loading charge of macrolide, loading a loading charge of the macrolide onto a bed of sorption resin, eluting with an eluent that contains at least one organic solvent selected from the group consisting of THF, acetonitrile, n-propyl alcohol, iso-propyl alcohol, ethyl alcohol, and acetone and water at a temperature greater than about 30° C., to about the boiling temperature of the solvent to obtain an effluent, collecting the main fraction of the effluent, and recovering the macrolide.

Preferred macrolides for the practice of the present invention include tacrolimus, ascomycin, sirolimus, everolimus, and pimecrolimus. When tacrolimus is the macrolide, the impurities reduced include at least ascomycin isomer of tacrolimus (RRT 1.19) and dihydrotacrolimus, quantification of which by HPLC is described hereinbelow. When ascomycin is the macrolide, the impurities reduced include at least tacrolimus. The macrolide used can be from any source.

The sorption resins useful in the practice of the present invention are well-known in the art and are preferably cross-linked, non-ionic styrene-divinyl benzene materials, but can be chemically modified. Acrylic-type sorption resins are also known. The sorption resins have highly porous structures whose surfaces can absorb—then desorb—various chemical species. The absorption and desorption are influenced by the environment, for example the solvent used. In the presence of polar solvents (e.g. water) the sorption resins exhibit hydrophobic behavior. When non-polar solvents are used (e.g. hydrocarbons), the sorption resins can exhibit some polar behavior. Typically, sorption resins have a macroreticular structure and have surface areas of at least about 300 m²/g.

Sorption resins useful in the practice of the present invention include the AMBERLITE® XAD resins available from Rohm and Haas; XAD 4, XAD 7 HP, XAD 16 HP, XAD 761, and XAD 1180, to mention just a few. Also useful are the DIAION® sorption resins available from Mitsubishi; HP 10, HP 20, BP 21, HP 30, HP 40, HP 50, SP 800, SP 825, SP 850, SP 875, SP 205, SP 206, SP 207, HP1MG and HP2MG, to mention just a few. AMBERLITE® XAD 1180 is an example of a preferred sorption resin for use in the practice of the present invention. AMBERLITE® XAD 1180 is a macroreticular crosslinked aromatic polymer. It is a non-ionic, hydrophobic, crosslinked polymer which derives its adsorptive properties from its patented macroreticular structure (containing both a continuous polymer phase and a continuous pore phase), high surface area, and the aromatic nature of its surface. Surface area is 500 m²/g or higher. Porosity is 0.60 ml/ml or higher. Product data sheet of PDS 0205 A-Jan.98-1/2 gives further information about this resin.

The loading charge can be provided as a solution of the macrolide in an organic solvent, or in an organic solvent combined with water, or as macrolide-loaded loading portion that is a macrolide which is adsorbed onto a loading portion of sorption resin.

When the loading charge of the macrolide is adsorbed onto (deposited onto) a loading portion of sorption resin prior to loading onto the bed of sorption resin, the adsorption includes preparing a solution of the macrolide in an organic solvent, optionally containing water, and combining the solution with a portion of sorption resin and water. The sorption resin can be the same as that used to prepare the bed, or it can be a different sorption resin. The loading portion of sorption resin can be about 33 percent to about 50 percent the volume of the bed. After the adsorption of the macrolide on the sorption resin is substantially complete, the loading charge is separated from the remaining solution. Separation can be by filtration. When the recirculating column method for making the loading charge is used, the column is simply decoupled from the recirculating system.

The organic solvent used to prepare the solution from which the loading charge is loaded or deposited is preferably selected from the group consisting of tetrahydrofuran (THF), acetone, acetonitrile (ACN), methanol, ethanol, n-butanol, n-propanol, iso-propanol, esters (e.g. ethyl acetate), and dipolar aprotic solvents, such as dimethylformamide (DMF). Most preferably, at ambient elution temperatures, the organic solvent is THF, acetone or ACN.

The addition of water reduces the solvent:water ratio and therefore increases the adsorption of macrolide on sorption resin.

The combination of the loading charge of the macrolide solution, loading portion of sorption resin, and water can be in any convenient vessel equipped with an agitator (e.g. a stirred-tank reactor).

By way of example, at ambient temperature, when the macrolide is tacrolimus, the solution can be about 100 g/l and the volume of anti-solvent can be at least about five times the volume of solution. However, at higher elution temperatures, the amount of solvent required for purification is reduced. The bulk volume of the loading portion of sorption resin can be approximately equal to the volume of solution. The skilled artisan will know to optimize the proportions by routine experimentation to obtain adsorption of the macrolide on the loading portion of the sorption resin.

In a subsequent step of this embodiment, the now macrolide-loaded loading portion is juxtaposed to a prepared bed of wet sorption resin. The bed is confined in a suitable vessel. Preferably, the bed is confined within a column, preferably of circular cross section. To prepare the bed, the desired amount of sorption resin is slurried with water or a mixture of water and a solvent (e.g., THF, ACN, methanol, acetone, etc.). A water-solvent combination is advantageous when the bed is to have a large diameter. The slurry is then transferred to the desired vessel, preferably a cylindrical column such as is used for column chromatography. The water (or water-solvent combination) is drawn-off to leave a bed of wet sorption resin. The practice of preparing and packing chromatography columns is well know to the skilled artisan and routiner alike, and the known practices are readily adapted to the practice of the present invention.

The loading portion can be juxtaposed to the bed of wet sorption resin simply as a layer thereon. When the loading charge is prepared in a recirculating system, the vessel containing the loading charge can be coupled to the container holding the bed of wet sorption resin by any means that establishes fluid communication therewith.

Separation of macrolide (e.g. tacrolimus, ascomycin, sirolimus, everolimus, or pimecrolimus) and impurities, whereby the level of impurities in the macrolide is reduced, is done by passing an eluent through the loading charge and subsequently through the bed of sorption resin juxtaposed thereto and in fluid communication therewith. Optionally, the eluent comprises an additional organic solvent selected from the group of solvents that are used for dissolving the macrolide in the first step of the process.

In the case that the loading charge is provided as a solution of the macrolide in an organic solvent, or in an organic solvent combined with water, the solution is injected into the prepared bed of wet sorption resin, the column is contacted with the flow of macrolide solution, the eluent is introduced into the stream of solution flowing through and around the loading portion of sorption resin, whereby the macrolide sample is gradually adsorbed onto the loading portion of sorption resin.

After the first elution, the bed may be placed in fluid communication with a second bed so that effluent from the first bed elutes through the second bed. After elution of first and second beds, the second bed may be, and, preferably, is decoupled from the first bed (i.e. fluid communication is broken) and elution is continued through the second bed alone. Optionally, the eluent is a mixture of THF and water having about 33 volume percent to 37. The eluent fractions may be collected and diluted with water, and thereafter may pass threw a third bed (column). Optionally, additional columns may be connected to the system and are diluted with additional amount of water in order to obtain a purer product. Preferably, additional amount of water is added to the last column in order to increase the adsorption of macrolide onto the sorption resin

The eluent includes water and an organic solvent at a temperature greater than about 30° C. to about the boiling temperature of the solvent. A preferred eluent, especially when tacrolimus is the macrolide, is essentially a mixture of THF and water having about 20 volume percent to about 50 volume percent, most preferably about 31 volume percent to about 40 volume percent, THF. When an organic solvent such as methanol, acetonitrile, acetone or n-butanol is used with the THF-water eluent, the THF content is less than 38 volume percent, preferably between about 4 and about 38 volume percent. Another preferred eluent is a mixture of acetonitrile and water having about 30 volume percent to about 70 volume percent, most preferably about 40 volume percent to about 65 volume percent, acetonitrile. When the eluent is a mixture of acetonitrile and water, the eluent can also include about 0.0005 to about 0.003 parts phosphoric acid to 1 part eluent. Again, the amount of solvent required is preferably between about 25 to about 35 percent at temperatures higher than ambient.

The effluent is eluted through the loading charge and bed of sorption resin juxtaposed thereto at a rate that depends on the gross cross sectional area of the bed (measured perpendicular to the flow of eluent). Preferably, the flow rate (relative to the cross sectional area) is less than about 25 cm/hour, preferably less than about 15 cm/hour. Lower elution rates increase the time, but improve the separation efficiency. A preferred elution rate for increased separation efficiency is of about 9 cm/hour to about 11 cm/hour.

The effluent flowing out of the bed of sorption resin (i.e. the effluent) is collected in one or more fractions, as in is customary to the skilled artisan using separation methods, like chromatography, that depend on preferential retention of chemical species on a stationary phase (e.g. a static bed).

The content and composition of the eluted fractions can be monitored by any convenient means. Detection and quantification of impurities in a macrolide, in particular ascomycin and dihydrotacrolimus in tacrolimus, can be carried-out by the hereinbelow described HPLC method.

Depending on, inter alia, column loading and the composition and flow rate of the eluent, the main fraction is collected, so that the final isolated product has about 0.1 area percent or less (by HPLC described below) of impurity ascomycin.

If desired, the macrolide separated from impurities and therefore having a reduced level of impurities can be isolated from effluent by any conventional means (e.g. extraction, lyophilization, evaporation, addition of anti-solvent). Water, alkanes and cycloalkanes can be mentioned as useful anti-solvents. Isolation methods can be combined. For example anti-solvent can be combined with concentrated eluent.

A preferred method of isolation includes concentration of the main fraction at 70° C. or less, preferably 60° C. or less, preferably at pressure of 760 mm Hg or less, to about 50 percent of its initial volume, whereby concentrated macrolide fraction is obtained. Phosphoric acid, about 1 to about 10 ml per liter of eluent, is preferably added before concentration to stabilize the macrolide.

Optionally, the concentrated main fraction is maintained at ambient temperature for a holding time. When a holding time is used, a preferred holding time is about 1-4 days.

Water immiscible solvent such as ethyl acetate or dichloromethane, and a base such as sodium hydroxide, an organic amine or ammonia solution are added to the concentrated macrolide fraction and the water immiscible solvent phase is separated and concentrated to obtain crystals of macrolide. The base is added until the pH is of about 9 or less.

The crystals of macrolide having reduced impurities are recovered by any conventional means, for example filtration (gravity or vacuum).

Further reduction in impurities can be achieved by subjecting the recovered product to several additional treatments such as crystallization and recrystallization.

The reduction in impurities in a macrolide accomplished by the method of the present invention can be monitored by the HPLC method described hereinbelow.

As discussed above, an elution temperature greater than about 30° C. to about the boiling temperature of the solvent may be used to improve the purification of macrolides, such as tacrolimus, ascomycin, sirolimus, everolimus and pimecrolimus, using adsorption resin chromatography.

The amount of organic solvent used in an eluent of solvent and water depends on the desired separation selectivity. As the concentration of organic solvent is increased, the separation selectivity decreases, such that above a certain limit, there is no separation selectivity during the elution process. Macrolides are not soluble in water, and have only a moderate solubility in organic solvent:water mixtures, where the organic solvent concentration is less than the separation selectivity limit discussed above.

In another aspect, the present invention relates to macrolides prepared by the method described above, especially tacrolimus, ascomycin, sirolimus (rapamycin), everolimus, and pimecrolimus.

Generally, elution temperatures greater than about 30° C. to about the boiling temperature of the solvent (included in the eluent) provide efficient purification of macrolides using adsorption resin chromatography, improving the purity of the final product and/or decreasing the amount of solvent required. The organic solvent content in the eluent is determined by the separation selectivity. Elution temperatures greater than about 30° C. to about the boiling temperature of the solvent results in a better separation selectivity and enables using greater amounts of solvent.

### Chromatographic Conditions Used for Examples

Column: ZORBAX SB-C18 75×4.6 mm; 3.5 μm

Pre-column: SymmetryShield RP18 3.9×20 mm; 5 μm

Eluent:

A: Measure 200 ml of acetonitrile into a 2000 ml volumetric flask, then dilute to volume with distilled water to 2000 ml total volume, followed by the addition of 100 μl of 50 percent acetic acid.

B: Add 100 μl of 50 percent acetic acid to 2000 ml of acetonitrile.

Table of gradients

| Time | Eluent “A” | Eluent “B” | Flow rate |
|---|---|---|---|
| (min) | (w/w %) | (w/w %) | (ml/min) |
| 0 | 60 | 40 | 2.3 |
| 15 | 55 | 45 | 2.3 |
| 25 | 30 | 70 | 1.8 |
| 25.1 | 60 | 40 | 1.8 |
| 27 | 60 | 40 | 1.8 |

Flow rate: 2.3 ml/min

Detection wavelength: 210 nm

Injected volume: 20 μl

Sample's solvent: acetonitrile

Temp. of column unit: 60° C.

Analysis time: 27 min

Retention time of tacrolimus: appr. 14 min

Retention times of impurities ascomycin (RRT 0.95), dihydrotacrolimus (RRT 1.25) and isomer of tacrolimus (RRT 1.19) are relative to tacrolimus and expressed as an area percent relative to the area of all peaks in the chromatogram.

### EXAMPLES

The results of a number of experiments, carried out under the conditions set forth above in accordance with the invention are provided in Table 1.

The starting substance of the experiments was tacrolimus crude product. Ten grams of the crude tacrolimus product was dissolved and adsorbed to an adsorption resin of AMBERLITE XAD 1180 in a 3.2 cm diameter column, having a height of 1 m. Ca. 610 ml of the adsorption resin was used for each experiment. The elution was carried out as set forth in the Table 1 at an elution rate of 90 ml/hour. The fractions were collected, where the volume of each fraction was 90 ml.

Several fractions were analyzed by the analytical method discussed above. Suitable fractions were combined, and a small amount of phosphoric acid was added to the combined fractions. The combined fractions were then concentrated under reduced pressure, removing the main part of the solvent content. (The concentrated volume was appr. ⅓ of the starting volume). A small amount of ammonium hydroxide solution was added to the concentrate, and the active substance of the concentrate was extracted with ethylacetate (¼ of the volume of the concentrate).

The solid content of the ethylacetate phase was established by evaporation of a small amount of solution to dryness under reduced pressure. The ethylacetate phase was concentrated under reduced pressure to 1.9 times the mass of the calculated solid mass. Cyclohexane, at 6 times the volume of the calculated solid mass, was added to the concentrate. Water, at 0.2 the times volume of the calculated solid mass, was added to the solution for ½ hour. Stirring was then applied for 1 hour at ambient temperature. The crystal-suspension was then kept at approximately 5° C. for approximately 20 hours. The crystals were filtered, and suspended with 100 ml n-hexane. The solid product was obtained after drying for at least 12 hours at not more than 70° C.

TABLE 1

| | | | | | | Isomer of | Dihidro- | |
|---|---|---|---|---|---|---|---|---|
| Number of | | | | Ascomycin | | tacrolimus | tacrolimus | Active |
| experiment | Eluent | Temperature | | (RRT 0.95) | Tacrolimus | (RRT 1.19) | (RRT 1.25) | substance |
| 1. | 30% THF | 45° C. | Crude | 0.7417 | 83.9148 | 7.2225 | 1.3548 | 8.52 g |
| | | | product | | | | | |
| | | | 00204 | | | | | |
| | | | Solid | 0.2478 | 91.6574 | 1.8831 | 0.9674 | 2.79 g |
| | | | product | | | | | |
| 2. | 30% THF | 60° C. | Crude | 0.7417 | 83.9148 | 7.2225 | 1.3548 | 8.52 g |
| | | | product | | | | | |
| | | | 00204 | | | | | |
| | | | Solid | 0.7148 | 92.0668 | 1.3745 | 1.1796 | 3.94 g |
| | | | product | | | | | |
| 3. | 32% THF | 45° C. | Crude | 0.7417 | 83.9148 | 7.2225 | 1.3548 | 8.52 g |
| | | | product | | | | | |
| | | | 00204 | | | | | |
| | | | Solid | 0.6128 | 89.4872 | 4.4016 | 1.0125 | 3.78 g |
| | | | product | | | | | |
| 4. | 28% THF | 45° C. | Crude | 0.7417 | 83.9148 | 7.2225 | 1.3548 | 8.52 g |
| | | | product | | | | | |
| | | | 00204 | | | | | |
| | | | Solid | 1.8706 | 86.1952 | 3.3482 | 0.8344 | 0.88 g |
| | | | product | | | | | |
| 5. | 34% THF | 35° C. | Crude | 0.7417 | 83.9148 | 7.2225 | 1.3548 | 8.52 g |
| | | | product | | | | | |
| | | | 00204 | | | | | |
| | | | Solid | 0.4671 | 93.9363 | 1.5955 | 1.0737 | 3.85 g |
| | | | product | | | | | |
| 6. | 34% THF | 40° C. | Crude | 0.7417 | 83.9148 | 7.2225 | 1.3548 | 8.52 g |
| | | | product | | | | | |
| | | | 00204 | | | | | |
| | | | Solid | 0.7522 | 95.6952 | 0.8919 | 1.2483 | 4.49 g |
| | | | product | | | | | |
| 7. | 32% THF | 35° C. | Crude | 0.7417 | 83.9148 | 7.2225 | 1.3548 | 8.52 g |
| | | | product | | | | | |
| | | | 00204 | | | | | |
| | | | Solid | 0.4541 | 94.6845 | 1.6111 | 0.9622 | 3.71 g |
| | | | product | | | | | |
| 8. | 34% THF | 20-25° C. | Crude | 0.7417 | 83.9148 | 7.2225 | 1.3548 | 8.52 g |
| | | | product | | | | | |
| | | | 00204 | | | | | |
| | | | Solid | 0.4278 | 92.4462 | 3.1671 | 0.8967 | 3.83 g |
| | | | product | | | | | |
| 9. | 31% THF | 40° C. | Crude | 0.7417 | 83.9148 | 7.2225 | 1.3548 | 8.52 g |
| | | | product | | | | | |
| | | | 00204 | | | | | |
| | | | Solid | 0.5528 | 94.4301 | 1.5222 | 0.8316 | 3.00 g |
| | | | product | | | | | |
| 10. | 32% THF | 40° C. | Crude | 0.7417 | 83.9148 | 7.2225 | 1.3548 | 8.52 g |
| | | | product | | | | | |
| | | | 00204 | | | | | |
| | | | Solid | 0.5092 | 87.5637 | 7.7093 | 0.7658 | 4.56 g |
| | | | product | | | | | |
| 11. | 33% THF | 40° C. | Crude | 0.7417 | 83.9148 | 7.2225 | 1.3548 | 8.52 g |
| | | | product | | | | | |
| | | | 00204 | | | | | |
| | | | Solid | 0.4345 | 87.7339 | 7.3422 | 0.8408 | 4.61 g |
| | | | product | | | | | |
| 12. | 30-40% | 45° C. | Crude | 0.7417 | 83.9148 | 7.2225 | 1.3548 | 8.52 g |
| | acetone | | product | | | | | |
| | | | 00204 | | | | | |
| | | | Solid | 0.5739 | 96.7339 | 0.4660 | 0.3805 | 2.09 g |
| | | | product | | | | | |
| 13. | 30% | 45° C. | Crude | 0.7417 | 83.9148 | 7.2225 | 1.3548 | 8.52 g |
| | i-propanol | | product | | | | | |
| | | | 00204 | | | | | |
| | | | Solid | 0.657 | 88.051 | 0.818 | 0.659 | 2.08 g |
| | | | product | | | | | |
| 14. | 40-60% | 45° C. | Crude | 0.7417 | 83.9148 | 7.2225 | 1.3548 | 8.52 g |
| | ethanol | | product | | | | | |
| | | | 00204 | | | | | |
| | | | Solid | 0.5037 | 90.5700 | 1.1586 | 0.6344 | 3.49 g |
| | | | product | | | | | |
| 15. | 25% | 45° C. | Crude | 0.7417 | 83.9148 | 7.2225 | 1.3548 | 8.52 g |
| | n-PrOH | | product | | | | | |
| | | | 00204 | | | | | |
| | | | Solid | 0.504 | 93.658 | 0.941 | 0.641 | 2.54 g |
| | | | product | | | | | |
| 16. | 45% | 45° C. | Crude | 0.7417 | 83.9148 | 7.2225 | 1.3548 | 8.52 g |
| | acetone | | product | | | | | |
| | | | 00204 | | | | | |
| | | | Solid | 0.559 | 88.528 | 2.513 | 0.775 | 2.43 g |
| | | | product | | | | | |
| 17. | 20% THF + 18% | 45° C. | Crude | 0.7417 | 83.9148 | 7.2225 | 1.3548 | 8.52 g |
| | acetone, | | product | | | | | |
| | from fr. 51 | | 00204 | | | | | |
| | 10.5% THF + 31.5% | | Solid | 0.341 | 95.687 | 0.887 | 0.648 | 3.21 g |
| | acetone | | product | | | | | |
| 18. | 20% THF + 18% | 45° C. | Crude | 0.7417 | 83.9148 | 7.2225 | 1.3548 | 8.52 g |
| | acetone, | | product | | | | | |
| | from fr. 61 | | 00204 | | | | | |
| | 10.5% THF + 31.5% | | Solid | 0.549 | 94.759 | 1.011 | 0.915 | 3.44 g |
| | acetone | | product | | | | | |
| 19. | 22% THF + 16% | 45° C. | Crude | 0.7417 | 83.9148 | 7.2225 | 1.3548 | 8.52 g |
| | acetone, | | product | | | | | |
| | from fr. 51 | | 00204 | | | | | |
| | 10.5% THF + 31.5% | | Solid | 0.258 | 94.426 | 0.738 | 0.809 | 3.07 g |
| | acetone | | product | | | | | |
| 20. | 22% THF + 16% | 45° C. | Crude | 0.7417 | 83.9148 | 7.2225 | 1.3548 | 8.52 g |
| | acetone, | | product | | | | | |
| | from fr. 61 | | 00204 | | | | | |
| | 10.5% THF + 31.5% | | Solid | 0.4180 | 95.7303 | 0.7751 | 0.8552 | 2.95 g |
| | acetone | | product | | | | | |
| 21. | 10% THF + 30% | 45° C. | Crude | 0.7417 | 83.9148 | 7.2225 | 1.3548 | 8.52 g |
| | acetone, | | product | | | | | |
| | from fr. 51 | | 00204 | | | | | |
| | 11% THF + 33% | | Solid | 0.3932 | 97.1666 | 0.5537 | 0.6227 | 3.06 g |
| | acetone | | product | | | | | |
| 22. | 11% THF + 33% | 45° C. | Crude | 0.7417 | 83.9148 | 7.2225 | 1.3548 | 8.52 g |
| | acetone | | product | | | | | |
| | | | 00204 | | | | | |
| | | | Solid | 0.496 | 94.669 | 0.841 | 0.820 | 3.05 g |
| | | | product | | | | | |
| 23. | 21% THF + 16% | 45° C. | Crude | 0.7417 | 83.9148 | 7.2225 | 1.3548 | 8.52 g |
| | acetone, | | product | | | | | |
| | from fr. 51 | | 00204 | | | | | |
| | 10.5% THF + 31.5% | | Solid | 0.546 | 94.994 | 0.874 | 0.865 | 3.44 g |
| | acetone | | product | | | | | |
| 24. | 21% THF + 16% | 45° C. | Crude | 0.7417 | 83.9148 | 7.2225 | 1.3548 | 8.52 g |
| | acetone, | | product | | | | | |
| | from fr. 51 | | 00204 | | | | | |
| | 10.5% THF + 33% | | Solid | 0.3628 | 93.9878 | 1.5673 | 0.7215 | 4.03 g |
| | acetone | | product | | | | | |
| 25. | 21% THF + 17% | 45° C. | Crude | 0.7417 | 83.9148 | 7.2225 | 1.3548 | 8.52 g |
| | acetone, | | product | | | | | |
| | from fr. 51 | | 00204 | | | | | |
| | 10.5% THF + 31.5% | | Solid | 0.2938 | 87.2596 | 4.5912 | 0.6588 | 3.34 g |
| | acetone | | product | | | | | |
| 26. | 21% THF + 17% | 45° C. | Crude | 0.7417 | 83.9148 | 7.2225 | 1.3548 | 8.52 g |
| | acetone, | | product | | | | | |
| | from fr. 51 | | 00204 | | | | | |
| | 10.5% THF + 33% | | Solid | 0.2980 | 95.1157 | 1.0149 | 0.7359 | 3.33 g |
| | acetone | | product | | | | | |
| 27. | 11% THF + 33% | 45° C. | Crude | 0.7417 | 83.9148 | 7.2225 | 1.3548 | 8.52 g |
| | acetone | | product | | | | | |
| | | | 00204 | | | | | |
| | | | Solid | 0.3850 | 89.8414 | 3.2984 | 0.8262 | 2.99 g |
| | | | product | | | | | |
| 28. | 21% THF + 15% | 45° C. | Crude | 0.7417 | 83.9148 | 7.2225 | 1.3548 | 8.52 g |
| | acetone, | | product | | | | | |
| | from fr. 51 | | 00204 | | | | | |
| | 10.5% THF + 33% | | Solid | 0.346 | 92.932 | 1.919 | 0.753 | 3.75 g |
| | acetone | | product | | | | | |

What is claimed is:

1. A process for purifying a macrolide, comprising:
a) providing a loading charge of a macrolide;
b) loading the loading charge of the macrolide onto a bed of sorption resin;
c) eluting with an eluent that contains at least one organic solvent selected from the group consisting of: THF, acetonitrile, n-propyl alcohol, iso-propyl alcohol, ethyl alcohol, and acetone and water at a temperature greater than about 30° C. to about the boiling temperature of the solvent to obtain an effluent;
d) collecting the main fraction of the effluent; and
e) recovering the macrolide.

2. The process of claim 1, wherein the macrolide is selected from the group consisting of: ascomycin, sirolimus, everolimus, and pimecrolimus.

3. The process of claim 1, wherein the sorption resin is AMBERLITE® XAD 1180.

4. The process of claim 1, wherein the loading charge is a solution of the macrolide in an organic solvent, or in an organic solvent combined with water.

5. The process of claim 1, wherein the loading charge is macrolide which is adsorbed onto a loading portion of sorption resin.

6. The process of claim 5, wherein the adsorption includes preparing a solution of macrolide in an organic solvent, combining the solution with a portion of sorption resin and water, and separating the adsorbed loading charge from the remaining solution.

7. The process of claim 6, wherein the portion of sorption resin is the same as that used to prepare the bed.

8. The process of claim 6, wherein the portion of sorption resin different from the sorption resin used to prepare the bed.

9. The process of claim 6, wherein the separation is by filtration.

10. The process of claim 6, wherein the organic solvent is selected from the group consisting of: tetrahydrofuran (THF), acetone, acetonitrile (ACN), methanol, ethanol, n-butanol, n-propanol, iso-propanol, esters (e.g. ethyl acetate), and dipolar aprotic solvents, such as dimethylformamide (DMF).

11. The process of claim 10, wherein the organic solvent is THF, acetone or acetonitrile.

12. The process of claim 1, wherein the bed of sorption resin is confined within a column.

13. The process of claim 1, wherein the eluent in step c) contains at least one organic solvent.

14. The process of claim 13, wherein the organic solvent is selected from the group consisting of: tetrahydrofuran (THF), acetone, acetonitrile (ACN), methanol, ethanol, n-butanol, n-propanol, iso-propanol, esters (e.g. ethyl acetate), and dipolar aprotic solvents, such as dimethylformamide (DMF).

15. The process of claim 1, wherein the eluent is a mixture of THF and water having about 20 volume percent to about 50 volume percent.

16. The process of claim 15, wherein the eluent is a mixture of THF and water having about 31 volume percent to about 40 volume percent.

17. The process of claim 1, wherein the eluent flow rate is less than about 25 cm/hour.

18. The process of claim 17, wherein the eluent flow rate is less 15 cm/hour.

19. The process of claim 18, wherein the eluent flow rate is of about 9 cm/hour to about 11 cm/hour.

20. The process of claim 1, wherein prior to step d) the bed is placed in fluid communication with a second bed of sorption resin.

21. The process of claim 20, wherein the second bed is decoupled from the first bed.

22. The process of claim 20, further comprising connecting additional beds of sorption resin to the system.

23. The process of claim 22, wherein additional amount of water is added to the last column.

24. The process of claim 1, wherein the recovering in step e) includes concentration of the main fraction in the presence of phosphoric acid at a temperature of about 70° C. or less at a pressure of about 760 mm Hg or less, adding water immiscible solvent and a base, separating the water immiscible solvent phase and concentrating it.

25. The process of claim 24, wherein the water immiscible solvent is ethyl acetate or dichloromethane.

26. The process of claim 24, wherein the base is selected from the group consisting of: sodium hydroxide, an organic amine or ammonia solution.
